(19) United States
(12) Patent Application Publication
(10) Pub. No.: US 2026/0314764 A1
(43) Pub. Date: Oct. 15, 2026

(54) Multi-Functional Insertable Nasal Device

(71) Applicant: Job Santoyo, Las Vegas, NV (US)

(72) Inventor: Job Santoyo, Las Vegas, NV (US)

(21) Appl. No.: 19/174,713

(22) Filed: Apr. 9, 2025

(51) Int. Cl. A44C 15/00; A61F 5/08; A61F 5/56

(52) U.S. Cl. CPC A44C 15/0065; A61F 5/08; A61F 5/56

(57) ABSTRACT

The multi-functional insertable nasal device includes a bridge with a bridge arch that fits adjacent to the columella (the visible forwardmost nose portion located between the nostrils). The bridge is connected on each end to a Y-shaped structure, the right and left Y-linkages. Each Y-linkage supports a base member and an apex member. The integrated components work together to change the shape of the nose (such as, for example, lifting the tip and/or narrowing the base), to reduce snoring, to lessen breathing difficulty, to emulate a nose ring without piercing the nasal septum, and/or to address other problems related to the nose.

### FIELD OF THE INVENTION

This invention relates generally to a nasal device that is inserted into the nose for adjusting the shape of the nose, reducing snoring, reducing breathing difficulty, and/or emulating a nose ring with no piercing of the nasal septum.

### BACKGROUND OF THE INVENTION

Many people have difficulty breathing through the nostrils, particularly while sleeping, which may lead to breathing through the mouth and snoring. Conventional nasal strips that fit on the exterior of the nose are available, but they are uncomfortable, hard to remove, may leave a residue on the nose after removal, and/or are expensive because they are disposable.

Other people have a desire to change the shape of the nose, such as to lift the tip of the nose for aesthetic reasons or to meet beauty conventions. One of the top cosmetic surgery procedures is rhinoplasty (also known as a “nose job”), which has been performed to reshape the nose since the 1800's. Just slightly lifting the tip of the nose can make an impactful change, so there is a desire for this cosmetic surgery, but the cost is prohibitive for many.

Some people like the appearance of a nose ring and desire to wear one, but they may not want to undergo a piercing of the septum of the nose. The reluctance to have a nasal septum piercing may be due to cost, health reasons, or restrictions against piercings imposed by others, such as employers or parents. Some people may not be good candidates for a septum piercing due to nose structural issues. Ideally, a septum piercing is placed in the alar cartilage, a softer cartilage that is commonly called the “sweet spot,” to avoid the tough lower cartilage of the middle of the nose. But the alar cartilage may not be suitable for piercing in people with a deviated septum or with a previous broken nose. For people who would enjoy having a nose ring but are prevented from undergoing a piercing of the septum, a non-piercing nose ring would be beneficial and desirable.

It would be helpful to provide a nasal device that addressed any of these three problems, but a nasal device that is multi-functional and could potentially address multiple problems would be even more efficient and advantageous. Accordingly, there is a need for an insertable multi-functional nasal device and method of use for adjusting the shape of the nose, reducing snoring, lessening breathing difficulty, and/or emulating a nose ring without piercing the nasal septum.

The discussion above is merely provided for general background information and is not intended to be used as an aid in determining the scope of the claimed subject matter.

### SUMMARY OF THE INVENTION

The inventive multi-functional nasal device, which can be inserted into the nose to tense the nose, open the nasal airway, and/or to lift the nose tip, includes a central bridge with an arch. When inserted into the forward portion of the nose, the arch fits adjacent to the columella (the visible nose portion at the forward end of the septum located between the nostrils). Two Y-shaped linkages extend from either side of the bridge. Each Y-linkage supports a base member and an apex member. The inventive nasal device is functional to address at least one or more of the following problems: to change the shape of the nose (lifting the tip and/or narrowing the base), to reduce snoring, to lessen breathing difficulty, and/or to emulate a nose ring without requiring a septum piercing.

By using the inventive nasal device, the shape of the nose is restructured without the expense of cosmetic surgery. Cosmetic surgery is not an option for many people, who may have a medical reason to avoid surgery, who may not want to undergo anesthesia, or who cannot afford the surgery. Thus, the inventive nasal device provides a beneficial option to change the nose shape without requiring any type of surgery. Further, restructuring through cosmetic surgery is not reversable, so even if the preferred aesthetic style for nose shape changes, the nose shape change achieved through cosmetic surgery would not be reversable without another surgery. When using the inventive nasal device, the change in shape is reversable, if desired.

The inventive nasal device provides support for the tip and side walls of the nose. This structural change in the nose shape assists in opening the airway passages. This may reduce breathing difficulty for some. The device is not expensive and is not invasive, so people with breathing difficulty can freely try it. The nasal device can be used without medication, or medication can be applied to one or more of the cushions disposed at the ends of the base members or apex members to potentially further reduce breathing difficulty.

Using the nasal device while sleeping provides support for the tip and side walls of the nose. This support encourages nasal breathing, which may reduce snoring.

The present invention is directed to a multi-functional insertable nasal device that may provide advantages in reducing snoring, easing breathing, lifting the tip of the nose, narrowing the base of the nose, reducing wrinkles, and/or allowing a person to wear a nose ring without the need for a piercing of the nasal septum.

In an aspect of the invention, the nasal device is formed integrally.

In an additional aspect of the invention, the nasal device is partially formed integrally with one or more of the cushions formed separately and fixedly attached.

In another aspect of the invention, the nasal device is painted or dyed a flesh color to minimize the visual appearance of the bridge portion of the nasal device that is exposed on the columella.

In a further aspect of the invention, the nasal device is configured with a decorative component on or near the bridge of the device to enhance the look of the nasal device, so the inventive nasal device more closely emulates a nose ring.

In the aspect of the invention that includes the decorative component, the decorative component may be comprised of one or more elements.

In the aspect of the invention that includes the decorative component, the decorative component may be fixedly attached or may be attachable and removable.

In a further aspect of the invention, the nasal device is reusable.

In another aspect of the invention, the nasal device is disposable.

In an additional aspect of the invention, the nasal device is formed of metal.

In a further aspect of the invention, the nasal device is formed of spring steel.

In another aspect of the invention, the nasal device is formed of plastic.

An object of the present invention is to provide a nasal device that can be inserted into the nose to provide one or more advantages, which include at least restructuring the nose, reducing snoring, diminishing difficulty in breathing, and/or allowing the user to display a nose ring without the user having to undergo piercing of the nasal septum.

These and other objects, features, and advantages of the present invention will become more readily apparent from the attached drawings and from the detailed description of the preferred embodiments which follow.

### BRIEF DESCRIPTION OF DRAWINGS

The preferred embodiments of the invention will hereinafter be described in conjunction with the appended drawings, provided to illustrate and not to limit the invention, where like designations denote like elements.

FIG. 1 is a front perspective view of an embodiment of the multi-functional insertable nasal device of the present invention.

FIG. 2 is a back perspective view of one embodiment of the present invention.

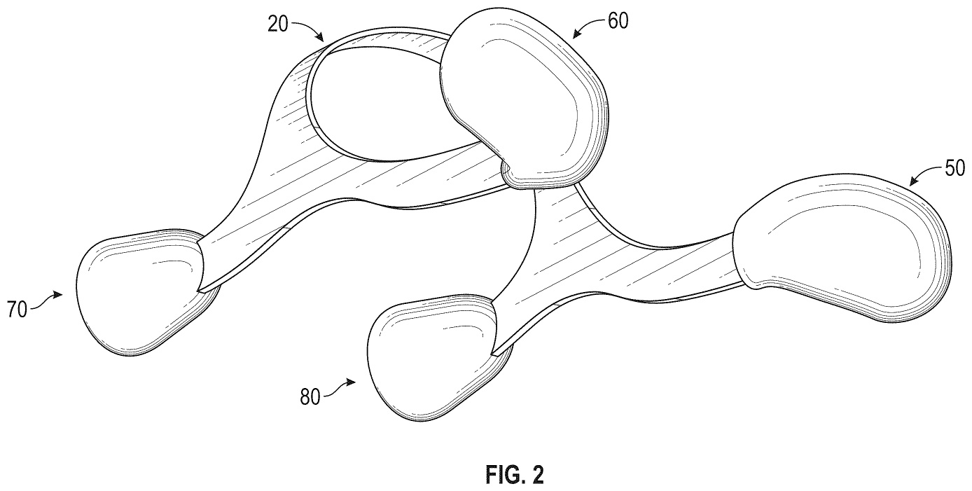

FIG. 3 is a front view of one embodiment of the present invention.

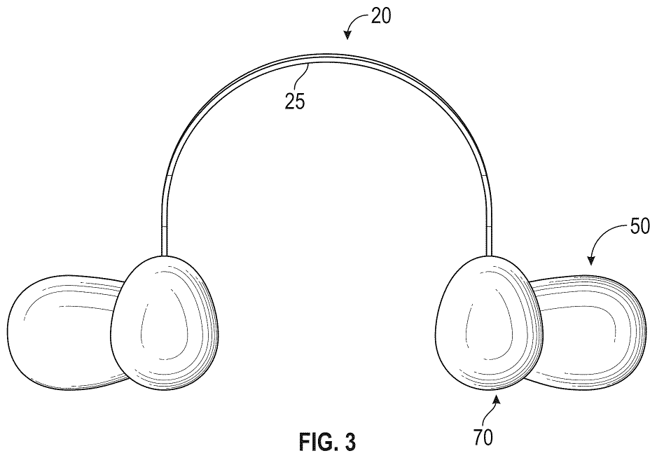

FIG. 4 is a back view of one embodiment of the present invention.

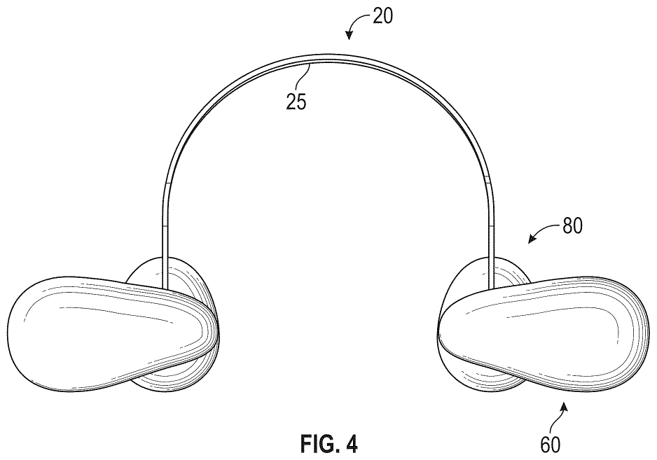

FIG. 5 is a left side view of one embodiment of the present invention.

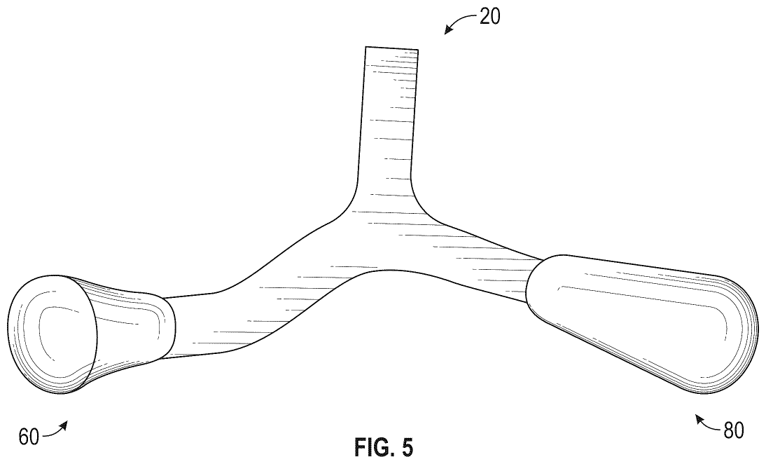

FIG. 6 is a right side view of one embodiment of the present invention.

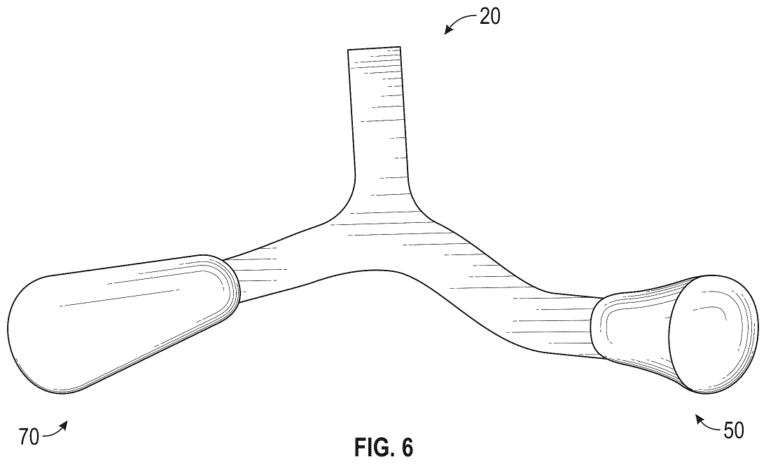

FIG. 7 is a top view of one embodiment of the present invention.

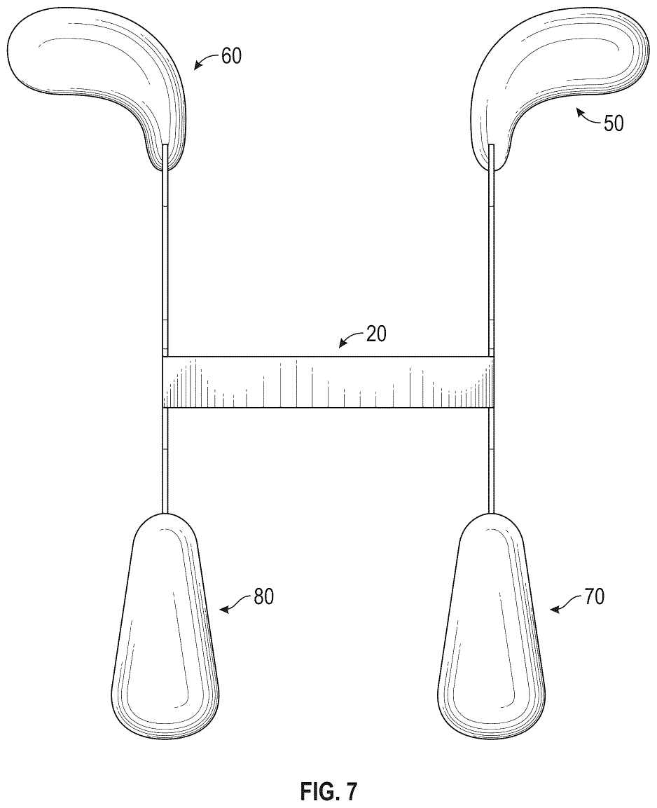

FIG. 8 is a bottom view of one embodiment of the present invention.

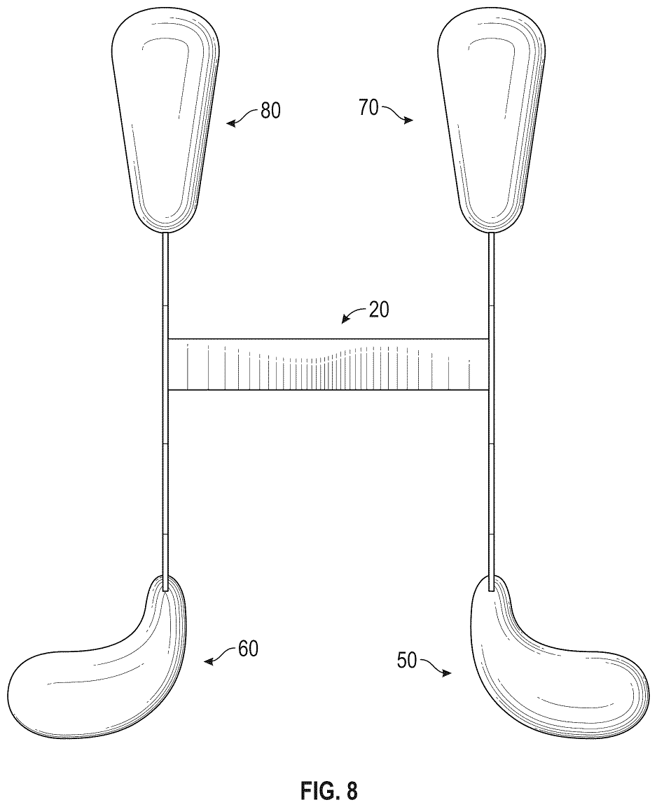

FIG. 9 is a side perspective view of a second embodiment of the present invention.

FIG. 10 is a right side view of an embodiment of the present invention being inserted into the nose.

FIG. 11 is a right side view of an embodiment of the present invention inserted into the nose.

Like reference numerals refer to like parts throughout the several views of the drawings.

### DETAILED DESCRIPTION OF THE INVENTION

Shown throughout the figures, the present invention is directed toward a multi-functional insertable nasal device that may be advantageous in one or more of the following use cases:
1. Causing the tip of the nose to be lifted to achieve a preferred nose shape;
2. Narrowing the base of the nose;
3. Reducing wrinkles in the skin around the base of the nose;
4. Reducing up to 50% of “smile” wrinkle lines;
5. Opening up the nasal cavity to enhance free air flow and aid in breathing;
6. Opening up the nasal cavity to reduce snoring;
7. Nasal administration of a medication to one or more specific areas of the nose;
8. Tensioning the skin on and around the nose; and/or
9. Imitating or emulating a nose ring without requiring the piercing of the nasal septum.

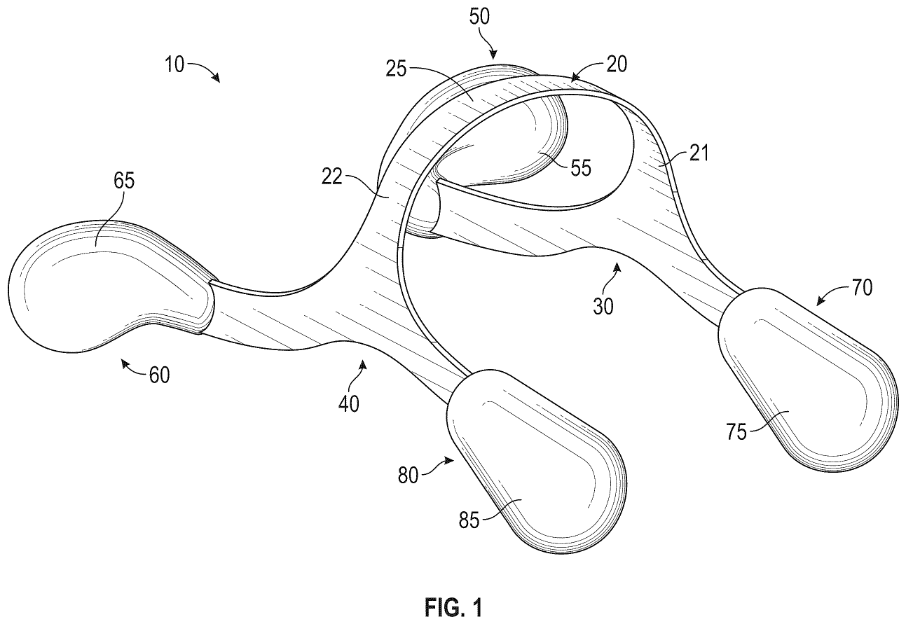

Referring now to FIG. 1, a multi-functional insertable nasal device, shown generally as reference number 10, is illustrated in accordance with a preferred embodiment of the present invention. As shown, in overview, the multi-functional insertable nasal device comprises a center bridge 20 comprising a bridge arch 25 that extends from a bridge right boundary 21 to a bridge left boundary 22. A right Y-linkage 30 is connected to the bridge 20 at the right boundary 21. A left Y-linkage 40 is connected to the bridge 20 at the left boundary 22. A right base 50 is connected to the right Y-linkage 30; the right base 50 preferably comprises a right base cushion 55 that, when inserted, will rest on the floor of the right nasal vestibule. A right apex 70 is connected to the right Y-linkage 30; the right apex 70 preferably comprises a right apex cushion 75 that, when inserted, will be positioned in the tip of the nose to the right of the nasal septum. A left base 60 is connected to the left Y-linkage 40; the left base 60 preferably comprises a left base cushion 65 that, when inserted, will rest on the floor of the left nasal vestibule. A left apex 80 is connected to the left Y-linkage 40; the left apex 80 preferably comprises a left apex cushion 85 that, when inserted, will be positioned in the tip of the nose to the left of the nasal septum.

In more detail, at the center of the multi-functional insertable nasal device is the bridge component 20 that forms the central connecting element for the entire symmetrical apparatus, connecting the right and left sides. This bridge 20 incorporates an arched design bridge arch 25 that extends from a bridge right boundary 21 to a bridge left boundary 22. This arch 25 mimics the natural curvature of nasal columella (the surface at the forward end of the nasal septum) to allow the arch 25 to sit comfortably against the columella.

Extending from both sides of the bridge 25 are specialized linking elements in a Y-configuration. The Y-linkages 30, 40 are in the shape of a Y-stem with two Y-arms extending in opposite directions. The right Y-linkage 30 comprises a Y-stem connected to the bridge 20 at the right boundary 21 and two right Y-arms extending in opposite directions. The left Y-linkage 40 comprises a Y-stem connected to the bridge 20 at the left boundary 22 and two left Y-arms extending in opposite directions. Thus, each Y-linkage 30, 40 branches from a stem portion into two arms. The two arms, when inserted into the nose, are an upwardly extending arm ending in an apex 70, 80 and a downwardly extending arm ending in a base 50, 60. The stem of the right Y-linkage 30 connects to the bridge at the bridge right boundary 21. The stem of the left Y-linkage 40 connects to the bridge at the bridge left boundary 22. These Y-shaped structures aid in the nasal device's overall form and functionality, allowing it to branch out to multiple contact points within the nasal passages.

The lower portions of each Y-linkage connect to base components. The right base 50 preferably includes a right base cushion 55. The left base 60 which preferably includes a left base cushion 65. These base elements provide stability and comfort during insertion and use. The base cushions 55, 65 provide comfort and minimize irritation when the nasal device is inserted so that the base cushions 55, 65 rest on the floor of the nasal vestibule between the base of the columella and the outer wall of the nose. The base cushions 55, 65 are preferably formed of a softer material than the other portions of the nasal device 10. The base cushions 55, 65 may be somewhat bulbous, as shown, and are preferably washable.

The upper portions of each Y-linkage connect to apex components. The right apex 70 preferably includes a right apex cushion 75. The left apex 80 preferably includes a left apex cushion 85. These apex elements 70, 80 function as the upper functional endpoints of the nasal device 10 and provide the structural support to lift the nose tip while potentially narrowing the base of the nose. This restructuring of the nose facilitates airflow in the upper nasal regions and reduces the limpness in that area that may result during periods of sleep. The apex cushions 75, 85 are preferably formed of a softer material than the other portions of the nasal device 10. The cushions 55, 65 may be somewhat bulbous, as shown, and are preferably washable.

The cushions 55, 65, 75, 85 provide contact points against the interior nasal surfaces. The cushions 55, 65, 75, 85 are preferably configured with smooth rounded edges for comfort. The shape of the cushions may be tear-shaped, as shown, or may be more spherical.

Though in the preferred embodiment, the right base cushion 55, right apex cushion 75, left base cushion 65, and left apex cushion 85 are formed of a material that is different (preferably softer or more cushiony) than the remainder of the device, in another embodiment, these cushions are formed of the same material as the remainder of the device. In this case, the cushioning is produced by the shape instead of by the material.

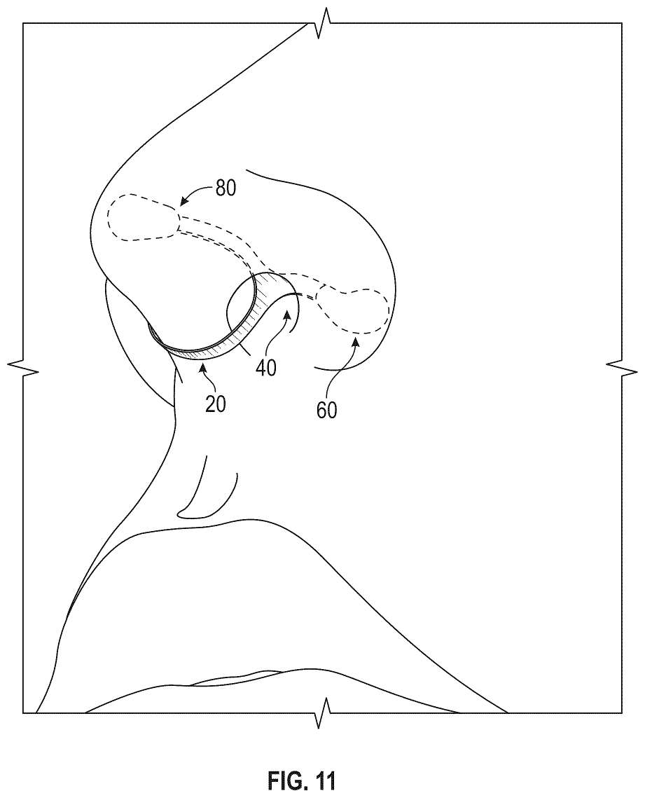

Preferably the right Y-linkage 30 is symmetrical to the left Y-linkage 40, though this symmetry is not necessary to the invention. In an embodiment, the insertable nasal device 10 may be customized to fit a particular nose, which can rest in variations between the right and left side of the device, in the angle or width of the bridge, in the length of the arms of the linkages 30, 40, and/or other lengths, widths, and angles. For example, the length of the arm of the right Y-linkage 30 that supports the base 50 need not be the same length of the arm of the right Y-linkage 30 that supports the right apex 70. This is exemplified in FIG. 11, in which the arm of the left Y-linkage 40 that supports the left apex 80 is longer than the arm of the left Y-linkage 40 that supports the left base 60. FIG. 11 also shows an exemplary positioning of the left apex 80 at the tip of the nose.

In an embodiment of the invention, the nasal device 10 is used to administer medications nasally via the very vascularized nasal mucosa. Nasal medication administration can be an alternative, non-invasive way to administer medications resulting in a quick onset of action that may be especially useful in the case of drugs metabolized in the gastrointestinal tract. The right base cushion 55, right apex cushion 75, left base cushion 65, and left apex cushion 85 each provide a contact point against the interior nasal mucosal surface. A medication can be applied to one or more of the cushions 55, 65, 75, 85. Upon insertion of the nasal device 10 into the nose, the medication can be absorbed through the nasal mucosa at the contact points.

In one embodiment of the invention, the insertable nasal device is formed integrally.

In another embodiment of the invention, the center bridge 20, right Y-linkage 30, left Y-linkage 40, right base 50, right apex 70, left base 60, and left apex 80 are formed integrally. In this embodiment, the right base cushion 55, right apex cushion 75, left base cushion 65, and left apex cushion 85 are formed separately and fixedly attached to the integrally formed structure.

In a further embodiment of the invention, the center bridge 20, right Y-linkage 30, left Y-linkage 40, right base 50, right apex 70, left base 60, left apex 80, right base cushion 55, and left base cushion 65 are formed integrally. In this embodiment the right apex cushion 75 and left apex cushion 85 are formed separately and fixedly attached to the integrally formed structure.

In yet another embodiment of the invention, the center bridge 20, right Y-linkage 30, left Y-linkage 40, right base 50, right apex 70, right apex cushion 75, left apex 80, and left apex cushion 85 are formed integrally. In this embodiment, the right base cushion 55 and left base cushion 65 are formed separately and fixedly attached to the integrally formed structure.

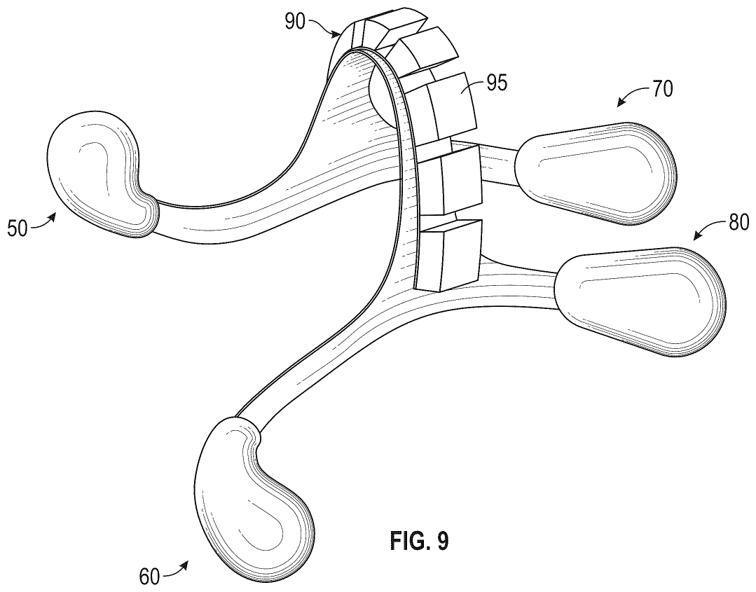

In the exemplary embodiment of FIG. 9, the bridge 25 is embellished with a decorative component 90. This embodiment is particularly designed for allowing the nasal device 10 to be used by the user as a nose ring. This is highly advantageous to the user, as the user achieves the look and effect of a nose ring but does not have to undergo piercing of the nasal septum.

The decorative component 90 may be a single element or multiple elements arranged in a desirable style and fashion as is known in the jewelry industry. The decorative component 90 may be subtle (such as, for example, a gold material or paint) or ostentatious (such as, for example, a chartreuse arch with turquoise drop beads hanging down toward the person's mouth). The decorative component 90 may be disposed on the bridge 25, on the bridge 25 and on one or both Y-linkages, or on one or both Y-linkages. The decorative component 90 may extend across the entire bridge, across a portion of the bridge, or be a single element positioned in the center of the bridge 25. An exemplary decorative component 90 is shown in FIG. 9, which is a row of rhinestones, but any ornamental element may be utilized as the decorative component 90. In another example, the decorative component is substantially flat (such as, for example, a colored paint, dye, film, or iridescent film), may be thin (such as, for example, an animal skin), may have a medium thickness (such as, for example, half pearls or rhinestones), may project substantially (such as, for example, a bone supported by the bridge or one or more beads), or may have projections or dangling elements (such as, for example, tiny hanging mirrors, fringe, or jewels on chains).

For example, the decorative component 90 may comprise crystals, rhinestones, ceramic or resin beads or particles, glass beads or particles, textiles, sequins, mirrors or pliable mirror foiling, links of chains, fur, dye, paint, precious stones, semiprecious stones, exotic skins, leathers including quilted or printed leathers, and other two-dimensional and three-dimensional synthetic (manufactured) or natural materials. The decorative component 90 may be formed of individual, linked, or composite elements, or a mixture thereof, fixedly attached to the nasal device 10. Individual elements 95 of the decorative component 90 may be set in individual settings or may be set in channels. The elements of the decorative component 90 may be of a consistent size or may vary in size. The decorative component 90 may be formed integrally with the other portions of the nasal device 10 or formed separately. The decorative component 90 may be fixedly attached to the nasal device 10, or all or part of the decorative component 90 may be detachable and reattachable.

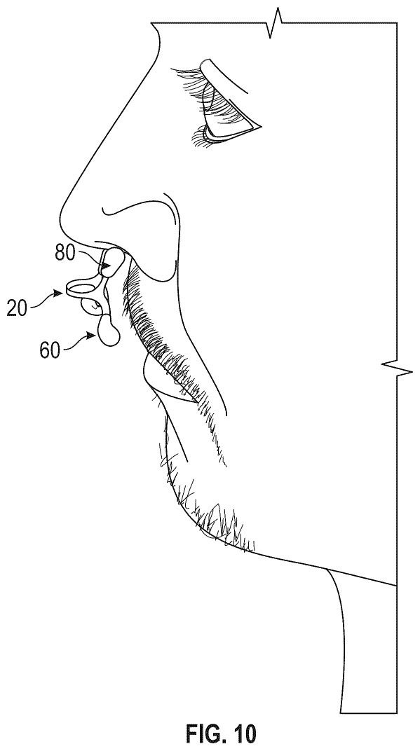

In the method of insertion of the nasal device 10, as seen in FIG. 10, the nasal device 10 is held with the right apex cushion 75 and left apex cushion 85 positioned upwardly and the base cushions 55, 65 positioned downwardly. The apex cushions 75, 85 are inserted into the tip of the nose. Then the bridge 25 is rotated slightly to bring it near or in contact with the columella while swinging the base elements 50, 60 toward the nose and positioning the base cushions 55, 65 onto the floor of the nose. The nasal device 10 may need to then be adjusted in position. For example, it may need to be wiggled to achieve more comfort, or it may need to be repositioned to achieve the elevation of the tip of the nose that is desired.

To remove the nasal device 10, the steps are generally reversed.

The nasal device is preferably made of a washable material, though a disposable nasal device 10 is within the scope of the invention. The nasal device 10 may be made of any suitable natural or manufactured material, such as plastic, wood products, or metal. Preferably the nasal device 10 is made of metal, and most preferably it is made of a spring metal, such as spring steel.

The multiple interconnected components work together to allow the nasal device to conform to the internal nasal anatomy while minimizing discomfort. The bridge 25 connecting the Y-linkages provides structural integrity while maintaining proper positioning once inserted. To increase comfort and stability, the nasal device 10 may be manufactured and offered in slightly smaller and slightly larger sizes to accommodate the diverse sizes of noses.

The present invention may suitably comprise, consist of, or consist essentially of the center bridge 20, right Y-linkage 30, left Y-linkage 40, right base 50, left base 60, right apex 70, and left apex 80.

The present invention may suitably comprise, consist of, or consist essentially of the center bridge 20, right Y-linkage 30, left Y-linkage 40, right base 50, left base 60, right apex 70, left apex 80, right base cushion 55, left base cushion 65, right apex cushion 75, and left apex cushion 85.

The present invention may suitably comprise, consist of, or consist essentially of the center bridge 20, right Y-linkage 30, left Y-linkage 40, right base 50, left base 60, right apex 70, left apex 80, right apex cushion 75, and left apex cushion 85.

The present invention may suitably comprise, consist of, or consist essentially of the center bridge 20, right Y-linkage 30, left Y-linkage 40, right base 50, left base 60, right apex 70, left apex 80, right base cushion 55, and left base cushion 65.

The multi-functional insertable nasal device 10 is configured to provide both functional efficacy and comfort during insertion and use. It provides support to the anatomical features of the nose to enable it to be used in any of the use cases disclosed herein.

The invention illustratively disclosed herein suitably may be practiced in the absence of any element which is not specifically disclosed herein.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

1. A nasal device, comprising:
a bridge comprising a bridge arch;
a right Y-linkage connected to the bridge;
a left Y-linkage connected to the bridge;
a right base connected to the right Y-linkage;
a right apex connected to the right Y-linkage;
a left base connected to the left Y-linkage; and
a left apex connected to the left Y-linkage;
wherein:
the bridge arch extends from a bridge right boundary to a bridge left boundary;
the right Y-linkage is disposed at the bridge right boundary;
the left Y-linkage is disposed at the bridge left boundary;
the right Y-linkage comprises a right Y-stem having a first end connected to the bridge at the bridge right boundary and having a second end from which two right Y-arms extend in opposite directions; and
the left Y-linkage comprises a left Y-stem having a first end connected to the bridge at the bridge left boundary and having a second end from which two left Y-arms extend in opposite directions.

2. (canceled)

3. (canceled)

4. The nasal device as recited in claim 1, wherein:
the right base comprises a right base cushion; and
the left base comprises a left base cushion.

5. The nasal device as recited in claim 1, wherein:
the right apex comprises a right apex cushion; and
the left apex comprises a left apex cushion.

6. The nasal device as recited in claim 1, wherein the bridge, the right Y-linkage, the left Y-linkage, the right base, the left base, the right apex, and the left apex are formed integrally from spring steel.

7. The nasal device as recited in claim 1, further comprising:
a right base cushion;
a left base cushion;
a right apex cushion; and
a left apex cushion;
wherein:
the bridge, the right Y-linkage, the left Y-linkage, the right base, the left base, the right apex, and the left apex are formed integrally; and
the right base cushion, the left base cushion, the right apex cushion, and the left apex cushion are formed separately and fixedly attached to the exterior of the distal ends of the right base, the left base, the right apex, and the left apex, respectively.

8. The nasal device as recited in claim 7, wherein:
the bridge, the right Y-linkage, the left Y-linkage, the right base, the left base, the right apex, and the left apex are formed integrally; and
the right base cushion, the left base cushion, the right apex cushion, and the left apex cushion are each tear-shaped or spherical.

9. The nasal device as recited in claim 1, wherein a decorative component is disposed on the bridge.

10. The nasal device as recited in claim 9, wherein the decorative component comprises a natural or manufactured stone.

11. (canceled)

12. (canceled)

13. (canceled)

14. A nasal device, comprising:
a bridge comprising a bridge arch, wherein the bridge arch extends from a bridge right boundary to a bridge left boundary;
a right Y-linkage comprising a right Y-stem disposed adjacent to at the bridge right boundary and comprising an upwardly-extending right Y-arm and a downwardly-extending right Y-arm;
a left Y-linkage comprising a left Y-stem disposed adjacent to the bridge left boundary and comprising an upwardly-extending left Y-arm and a downwardly-extending left Y-arm;
a right base disposed at the distal end of the downwardly-extending right Y-arm;
a right apex disposed at the distal end of the upwardly-extending right Y-arm;
a left base disposed at the distal end of the downwardly-extending left Y-arm;
a left apex disposed at the distal end of the upwardly-extending left Y-arm;
wherein the upwardly-extending right Y-arm is shorter than the downwardly-extending right Y-arm; and
wherein the upwardly-extending left Y-arm is shorter than the downwardly-extending left Y-arm; whereby, when inserted, the left apex and right apex are positioned at the tip of the nose to elevate the tip of the nose upward.

15. The nasal device as recited in claim 14, wherein:
the right base cushion, the left base cushion, the right apex cushion, and the left apex cushion are formed separately and fixedly attached to the exterior of the right base, the left base, the right apex, and the left apex, respectively.

16. The nasal device as recited in claim 14, wherein:
the right base comprises a right base cushion;
the left base comprises a left base cushion;
the right apex comprises a right apex cushion; and
the left apex comprises a left apex cushion; and
wherein the right base cushion, the left base cushion, the right apex cushion, and the left apex cushion are each bulbous and tear-shaped or spherical.

17. The nasal device as recited in claim 14, wherein:
the bridge, the right Y-linkage, the left Y-linkage, the right base, the left base, the right apex, and the left apex are formed integrally; and
the right base cushion, the left base cushion, the right apex cushion, and the left apex cushion are formed separately and fixedly attached to the exterior of the distal ends of the right base, the left base, the right apex, and the left apex, respectively.

18. The nasal device as recited in claim 14, wherein a decorative component is disposed on the bridge.

19. The nasal device as recited in claim 18, wherein the decorative component comprises a natural or manufactured stone.

20. The nasal device as recited in claim 18, wherein the decorative component comprises a paint or dye.

21. The nasal device as recited in claim 14, wherein the bridge, the right Y-linkage, the left Y-linkage, the right base, the left base, the right apex, and the left apex are formed integrally from spring steel.
